(19) United States
(12) Patent Application Publication
(10) Pub. No.: US 2025/0186197 A1
(43) Pub. Date: Jun. 12, 2025

(54) BIOLOGICAL VALVE MATERIAL BASED ON ALDEHYDE GROUP CROSSLINKING, PREPARATION METHOD THEREFOR, AND USE THEREOF

(71) Applicant: SICHUAN UNIVERSITY, Chengdu (CN)

(72) Inventors: Yunbing Wang, Chengdu (CN); Cheng Zheng, Chengdu (CN); Li Yang, Chengdu (CN); Gaocan Li, Chengdu (CN); Rifang Luo, Chengdu (CN); Dajun Kuang, Chengdu (CN); Caili Ma, Chengdu (CN); Xueyu Huang, Chengdu (CN)

(21) Appl. No.: 19/060,791

(22) Filed: Feb. 24, 2025

Related U.S. Application Data
(63) Continuation-in-part of application No. PCT/CN2022/132873, filed on Nov. 18, 2022.

(30) Foreign Application Priority Data
Nov. 15, 2022 (CN) 202211431077.5

(51) Int. Cl. A61F 2/24; A61L 27/26

(52) U.S. Cl. CPC A61F 2/2415; A61L 27/26; A61F 2240/001

(57) ABSTRACT

Disclosed are a biological valve material based on aldehyde group crosslinking, a preparation method therefor, and use thereof. The preparation method includes: step S110, contacting the biological material with an aldehyde group crosslinking agent solution for crosslinking; step S120, soaking the biological material treated in step S110 in a solution containing a first functional monomer to chemically introduce first carbon-carbon double bonds; wherein the first functional monomer has the first carbon-carbon double bonds and an ethylene oxide group; step S130, soaking the biological material treated in step S120 in a solution containing a second functional monomer, wherein the second functional monomer has second carbon-carbon double bonds; and step S200, performing a polymerization reaction of the carbon-carbon double bonds under an action of an initiator to obtain a biological valve material. The method yields more and larger polymer crosslinking networks by means of two rounds of crosslinking.

### CROSS-REFERENCE TO RELATED APPLICATIONS

The present disclosure is a Continuation-In-Part Application of PCT Application No. PCT/CN2022/132873, filed on Nov. 18, 2022, which claims the priority of Chinese Patent disclosure No. 202211431077.5, filed on Nov. 15, 2022, the entire contents of which are hereby incorporated by reference.

### TECHNICAL FIELD

The present invention relates to the technical field of interventional materials, and in particular to a biological valve material based on aldehyde group crosslinking, a preparation method therefor, and use thereof.

### BACKGROUND

Biological heart valves are generally prepared from the porcine or bovine pericardium after glutaraldehyde crosslinking and used in valvular heart disease treatment to replace damaged autologous heart valves. Compared with mechanical heart valves, biological heart valves have a series of advantages: biological heart valves have excellent fluid mechanics properties which are much closer to native heart valves than mechanical valves; biological heart valves are less thrombogenic than mechanical valves, and patients do not need lifelong anticoagulation treatment after implantation; biological heart valves are compressible and can be implanted by mean of minimally invasive interventional surgery, thereby avoiding the need for thoracotomy procedures and reducing the damage of valve replacement to patients. These advantages allow the biological heart valve in clinical disclosure to increase year by year and become the mainstream prosthetic valve.

At present, almost all biological heart valve products used in clinic are prepared after glutaraldehyde crosslinking, which can improve the mechanical properties of the pericardium and reduce its immunogenicity to a certain extent. However, glutaraldehyde crosslinked biological valves still face the problems of low stability and crosslinking degree. This will lead to component degradation and destruction of the valve after implantation, damaging its structural integrity and compromising structural integrity, ultimately resulting in structural degeneration and failure. Therefore, the stability and crosslinking degree of the valve still need to be further improved. In addition, the degradation of biological valve components will further induce mechanical damage and accelerate calcification and structural degeneration to its leaflets, thereby affecting the normal blood fluid performance of the biological heart valve and reducing its service life.

Currently, glutaraldehyde crosslinked biological heart valves are still the mainstream biological heart valves in clinical use. In view of the fact that glutaraldehyde crosslinked biological heart valves still have problems with low stability and crosslinking degree, as well as facing the risk of structural degeneration and failure caused by structural degradation and destruction, further development of valves based on glutaraldehyde crosslinking is not only in line with the actual production needs but also of great significance for scientific research.

The applicant of the present disclosure has long been committed to the research of biological heart valves. For example, in the previous research, the Chinese patent publication No. CN 114748694A disclosed a co-crosslinked biological valve material, preparation method therefor, and use thereof, in which the biological valve material was functionally modified by introducing functional monomers for co-crosslinking at the same time of crosslinking treatment; and in the biological valve preparation methods disclosed in Chinese patent publication Nos. CN 114748693A, CN 114748697A, CN 114748696A and CN 114748695A, the functional monomer was added to the functional monomer and the carbon-carbon double bonds were introduced into the functional monomer as the basis for further crosslinking, and the modification of biological valve material was completed through two rounds of crosslinking.

In the aforementioned research, whether it is introducing functional monomers for co-crosslinking modification through glutaraldehyde crosslinking, or introducing carbon-carbon double bonds as the basis for further crosslinking during the co-crosslinking process, new modified substances are introduced during the glutaraldehyde crosslinking process to participate in the crosslinking reaction.

### SUMMARY

The present disclosure provides a biological valve material based on aldehyde group crosslinking, preparation method therefor, and use thereof. Without changing the conventional glutaraldehyde crosslinking reaction, the carbon-carbon double bonds are used as the basis for secondary crosslinking, providing the glutaraldehyde crosslinked valve with a controllable chance and range of crosslinking.

A method for preparing a biological valve material based on aldehyde group crosslinking, including:

Step S110: contacting the biomaterial with an aldehyde group crosslinking agent solution for crosslinking;

Step S120: soaking the biomaterial treated in step S110 in a solution containing a first functional monomer to chemically introduce a first carbon-carbon double bond; the first functional monomer has the first carbon-carbon double bond and an ethylene oxide group;

Step S130: soaking the biomaterial treated in step S120 in a solution containing a second functional monomer, wherein the second functional monomer has a second carbon-carbon double bond; and

Step S200, performing a polymerization reaction of the carbon-carbon double bonds to obtain the biological valve material under the action of an initiator.

Optionally, the aldehyde group crosslinking agent is glutaraldehyde or formaldehyde.

Optionally, the biological material is an animal tissue, and the animal tissue is selected from one or more of the following: pericardium, valve, intestinal valve, meninges, lung valve, blood vessel, skin or ligament.

Optionally, the animal tissue is a fresh animal tissue or a decellularized biological tissue.

The step S200 includes:

Optionally, adding the initiator to a system treated in a previous step; or taking the biomaterial treated in the previous step out and directly or after washing soaking the biomaterial treated in the previous step in a solution containing the initiator.

Optionally, the initiator is a single initiator or a mixed initiator.

Optionally, the mixed initiator is:

a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and a mixture of tetramethylethylenediamine, or a mixture of sodium persulfate and tetramethylethylenediamine; and the concentration of each component in the mixture is in the range of 1 to 100 mM.

Optionally, the single initiator is any component of each mixed initiator.

Optionally, the first functional monomer is selected from at least one of the following: allyl glycidyl ether, glycidyl methacrylate and glycidyl acrylate.

Optionally, the second functional monomer is selected from one or more of the following: polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-vinylbisacrylamide, (ethane-1,2-diylbis(oxy))bis(ethane-2,1-diyl) diacrylate, N,N′-dimethylacrylamide, N,N-dimethylmethacrylamide, and double-bond polylysine.

In step S110:

Optionally, the w/w concentration of the aldehyde group crosslinking agent solution is in the range of 0.1% to 5%; and the crosslinking time is in the range of 0.5 h to 120 h.

In step S120:

the w/w concentration of the first functional monomer in the solution containing the first functional monomer is in the range of 1% to 10%; and the chemical reaction time is in the range of 2 h to 120 h.

Optionally, the solution containing the first functional monomer only contains the first functional monomer and a solvent that does not participate in the chemical reaction.

Optionally, the solvent in the solution containing the first functional monomer is one or more of the following: water, physiological saline, a neutral pH buffer, and an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol.

In step S130:

the v/v concentration of the second functional monomer in the solution containing the second functional monomer is in the range of 0.1% to 20%; and the immersion time is in the range of 0.5 h to 120 h.

the v/v concentration of the second functional monomer in the solution containing the second functional monomer is in the range of 0.1% to 6%.

Optionally, the second functional monomer permeates the biomaterial by physical penetration.

The physical penetration may be understood as when the biomaterial treated in step S120 is soaked in a solution containing the second functional monomer, the second functional monomer in the solution adheres to the surface of the biomaterial or embeds into the gaps in the biomaterial. During this process, no chemical reaction occurs between the second functional monomer and the biomaterial.

Optionally, the solution containing the second functional monomer only contains the second functional monomer and a solvent that does not participate in the reaction.

Optionally, the solvent in the solution containing the second functional monomer is one or a mixture of the following: water, physiological saline, ethanol, isopropanol or a neutral pH buffer solution.

The present disclosure further provides a biological valve material prepared by the preparation method.

The present disclosure further provides a biological valve material, including:

Step S110: contacting the biomaterial with an aldehyde group crosslinking agent solution for crosslinking;

Step S120: soaking the biomaterial treated in step S110 in a solution containing a first functional monomer to chemically introduce a first carbon-carbon double bond; wherein the first functional monomer has the first carbon-carbon double bond and an ethylene oxide group;

Step S130: soaking the biomaterial treated in step S120 in a solution containing a second functional monomer, wherein the second functional monomer has the second carbon-carbon double bond; and

Step S200, performing a polymerization reaction of the carbon-carbon double bonds to obtain a biological valve material under the action of an initiator.

The present disclosure further provides a biological valve, including a stent and leaflets, wherein the leaflets are made of the biological valve material.

Optionally, the biological valve is a prosthetic heart valve.

The present disclosure further provides an interventional system, including a prosthetic heart valve and a catheter assembly, wherein the prosthetic heart valve is delivered by the catheter assembly after being folded, and the prosthetic heart valve includes a stent and leaflets, and the leaflets are made of the biological valve material.

Compared with the prior art, this disclosure has at least the following beneficial effects:

(1) Based on the biological valve material crosslinked by glutaraldehyde, in the present disclosure, double bonds are introduced to the glutaraldehyde crosslinked biological valve material through double-bond modification as the basis for secondary crosslinking, and then a polymerization of the double bonds on the glutaraldehyde crosslinked biological valve material and the double bonds of the functional monomers is initiated for secondary crosslinking, thereby introducing the polymeric crosslinked network of the functional monomers, which can further increase the crosslinking degree of the biological valve material.

(2) The present disclosure introduces carbon-carbon double bonds to the glutaraldehyde crosslinked biological valve material, and further initiates the polymerization between the carbon-carbon double bonds on the double-bonded biological valve material and the carbon-carbon double bonds of the functional monomer to introduce a polymeric crosslinked network of functional monomer, which can further reduce the binding of collagenase in the human body to the collagen matrix on the biological valve material through physical blocking to a certain extent, protecting the collagen matrix of the biological valve material and improving the stability of the glutaraldehyde crosslinked biological valve material, and further reducing calcification risk caused by structural degradation of the biological valve material, so the biological valve material also has certain anti-calcification properties.

(3) The present disclosure introduces carbon-carbon double bonds to the glutaraldehyde crosslinked biological valve material, and further initiates the polymerization between the carbon-carbon double bonds on the glutaraldehyde crosslinked biological valve material and the carbon-carbon double bonds of the functional monomer to introduce a polymeric crosslinked network of the functional monomers, which can serve as a polymeric barrier to further reduce the binding of calcium ions in the environment to the mineralized zone on the biological valve material that are prone to binding with calcium ions, reducing the risk of calcification and thus playing an anti-calcification role.

(4) The present disclosure introduces carbon-carbon double bonds to the glutaraldehyde crosslinked biological valve material, and further initiates the polymerization between the carbon-carbon double bonds on the double-bond glutaraldehyde crosslinked biological valve material and the carbon-carbon double bonds of the functional monomer to introduce a polymeric crosslinked network of functional monomer, thereby increasing the crosslinking degree of the biological valve material and thus making the structure of the biological valve material more rigid while increasing the elasticity; moreover, the polymeric crosslinked network of functional monomer fills the gaps between the collagen matrices on the biological valve material to inhibit the deformation of collagen fibers, making the biological valve material harder while increasing the elasticity.

(5) Compared with the applicant's previous research on methods for modifying biological valve materials that introduced the carbon-carbon double bonds by adding functional monomers for co-crosslinking during glutaraldehyde modification, in the process of modifying the biological valve materials of the present disclosure, the glutaraldehyde crosslinking treatment is firstly performed and then the residual amino groups and active groups such as hydroxyl and carboxyl groups on the glutaraldehyde crosslinked valve are chemically conjugated to the functional monomers having carbon- carbon double bonds. The functional monomers with carbon-carbon double bonds undergo chemical reactions with the amino groups, hydroxyl groups and carboxyl groups on the surface of glutaraldehyde crosslinked valve through the ethylene oxide group, and the carbon-carbon double bonds are mainly grafted on the surface of the biological valve material. As there is no other substance that can participate in the crosslinking reaction during the process of glutaraldehyde crosslinking modification of the biological valve material, the original fiber structure of the biomaterial can be better protected. This can effectively ensure the mechanical properties of the valve while maintaining the orientation direction of the original fibers of the biomaterial, thereby avoiding the problem of directly adding double-bond functional monomers during crosslinking in previous research, which may damage the original fiber orientation of the biomaterial and increase fiber disorder.

(6) On the basis of chemically grafting the first carbon-carbon double bond, the second carbon-carbon double bond is further introduced through physical penetration, providing more double bonds as a crosslinking basis for secondary crosslinking, which can further increase the crosslinking degree of the biological valve material and improve the mechanical properties of the biological valve material.

### BRIEF DESCRIPTION OF DRAWINGS

FIG. 1 is a process flow chart of an implementation method of crosslinking after double-bond copolymerization of the present disclosure;

FIG. 2 is a reaction schematic diagram of an implementation method of crosslinking after double-bond copolymerization of the present disclosure;

FIG. 3 is an image showing an alizarin red staining result of the control group 1 (glutaraldehyde crosslinked porcine pericardium) after subcutaneous implantation in a rat for 30 days;

FIG. 4 is an image showing an alizarin red staining result of sample 1 of Example 1 after subcutaneous implantation in a rat for 30 days;

FIG. 5 is an image showing an alizarin red staining result of sample 2 of Example 2 after subcutaneous implantation in a rat for 30 days;

FIG. 6 is an image showing an alizarin red staining result of sample 5 of Example 5 after subcutaneous implantation in a rat for 30 days;

FIG. 7 is a schematic diagram of a prosthetic heart valve of the present disclosure;

FIG. 8 is a schematic diagram of an intervention system of the present disclosure.

### DESCRIPTION OF EMBODIMENTS

The technical solutions according to the embodiments of the present disclosure will be described clearly and fully in combination with the drawings according to the embodiments of the present disclosure. Apparently, the described embodiments are not all embodiments of the present disclosure, but only part of the embodiments of the present disclosure. Based on the disclosed embodiments, all other embodiments obtained by those skilled in the art without creative work fall into the scope of this invention.

Unless otherwise defined, all technical and scientific terms used herein have the same meaning as commonly understood by a person skilled in the art. The terms in the description of the present disclosure are used to describe specific embodiments, and not to limit the present disclosure.

Almost all existing biological valve products used in clinical implantation are made of glutaraldehyde crosslinked biological valve materials. Glutaraldehyde reacts with collagen in the biological valve material, crosslinking the collagen matrix and reducing the immunogenicity of the material while enhancing its mechanical strength. However, the biological valve materials often exhibit low crosslinking degrees after glutaraldehyde crosslinking, making them susceptible to structural degradation. This leads to component degradation and compromises structural integrity, ultimately resulting in structural degeneration and failure. Additionally, degradation of biological valve components may in turn cause mechanical damage to valve leaflet structures and promote calcification, impeding the normal opening and closing movement of the valve and reducing the lifespan of the biological valve with the structural degeneration.

Currently, glutaraldehyde crosslinked biological heart valves are still the mainstream biological heart valves in clinical use. In view of the fact that glutaraldehyde crosslinked biological heart valves still have problems with low stability and crosslinking degree, as well as facing the risk of structural degeneration and failure caused by structural degradation and destruction, a series of further crosslinking and modification of valves based on glutaraldehyde crosslinking is not only in line with the actual production needs, but also of great significance for scientific research.

Therefore, based on the glutaraldehyde crosslinked biological valve material, the present disclosure introduces carbon-carbon double bonds on the biological heart valve material as a platform for secondary crosslinking by double-bond treatment of the glutaraldehyde crosslinked biological valve material. By initiating a copolymerization reaction between the double bonds in the double-bond glutaraldehyde crosslinked biological valve material and the double bonds of the functional monomers, a polymeric network of functional monomers is introduced to the glutaraldehyde crosslinked biological valve material, thereby further expanding the crosslinked network. That is, on the basis of scheme one, a second carbon-carbon double bond is further introduced by physical penetration of a second functional monomer (containing the second carbon-carbon double bond), which will increase the crosslinking degree of the glutaraldehyde crosslinked biological valve material valve, enhance its structural stability, and further reduce the degree of calcification of the material to improve its anti-calcification performance.

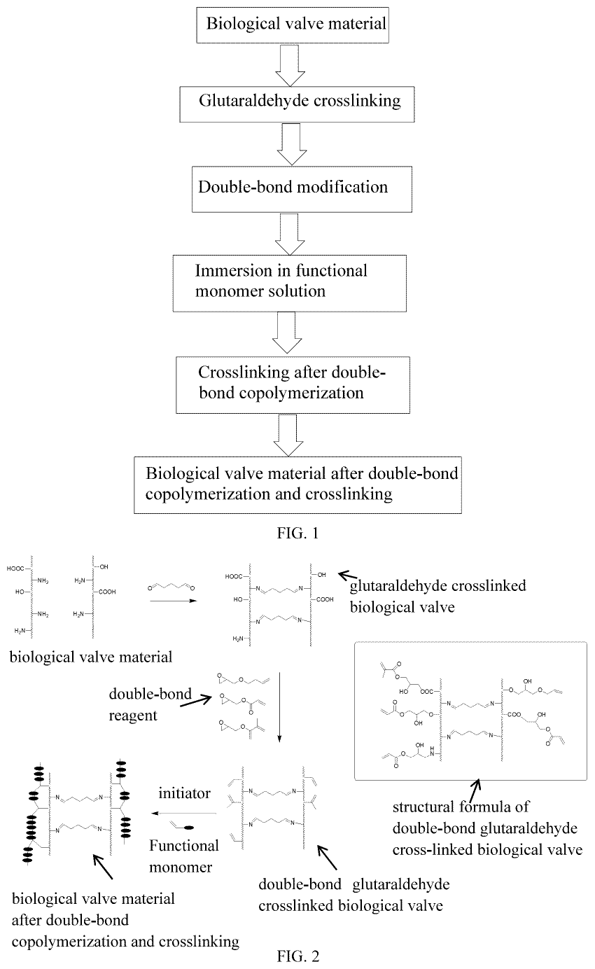

Specifically, it includes (see FIG. 1):

S110: soaking the biological valve material in an aldehyde group crosslinking agent solution for crosslinking to prepare a glutaraldehyde crosslinked biological valve material;

S120: soaking the glutaraldehyde crosslinked biological valve material prepared in step S110 in a solution of a double-bond reagent (first functional monomer) for double-bond modification to prepare a double-bond biological valve material; wherein the double-bond reagent (first functional monomer) has at least one first carbon-carbon double bond and an ethylene oxide group.

S130: soaking the double-bond biological valve material obtained in step S120 with a functional monomer (second functional monomer) solution, wherein the second functional monomer has at least one second carbon-carbon double bond; and

S200: adding an initiator to the solution after the soaking in step S130 to contact with the biological valve material and the functional monomer solution to initiate double-bond polymerization.

In the present disclosure, the biomaterial first undergoes a crosslinking reaction with an aldehyde group crosslinking agent (S110), then reacts with the active group of the first functional monomer to introduce to the first carbon-carbon double bond (S120), and afterward, the second carbon-carbon double bond is introduced through physical penetration of the second functional monomer (S130). During the preparation process, the aldehyde group crosslinking agent is first added to react with part of the amino groups of the biomaterial, and then the first functional monomer is added so that the first carbon-carbon double bond is directly grafted by applying the remaining amino groups and other groups (such as hydroxyl and carboxyl groups) on the biological material to react with the active group of the first functional monomer. The active group of the first functional monomer is an ethylene oxide group. In addition to the remaining amino groups participating in the reaction, the hydroxyl and carboxyl groups of the biomaterial can also react with the ethylene oxide group to participate in the chemical reaction. On the basis of the chemical reaction to introduce the first carbon-carbon double bond, the second carbon-carbon double bond is then introduced through physical penetration of the second functional monomer. Finally, the first carbon-carbon double bond introduced by chemical reaction is further polymerized under the action of an initiator to form a crosslinked network, which improves the anti-coagulation, anti-calcification, elasticity and other properties of the biological valve based on glutaraldehyde crosslinking.

The schematic diagram of this disclosure is explained as follows:

In this double-bond crosslinking scheme, after the biological valve material is crosslinked with glutaraldehyde, the first carbon-carbon double bond is further introduced by using a double-bond reagent (first functional monomer) solution for double-bonding the glutaraldehyde crosslinked biological valve material, which is then used as a platform for secondary crosslinking. The double-bond reagent (first functional monomer) used has both the carbon-carbon double bond and the ethylene oxide group.

To facilitate understanding of the chemical principles involved in this scheme, FIG. 2 is illustrated as an example for explanation: The first functional monomer (i.e., the double bonding reagent) is used to modify the glutaraldehyde crosslinked biological valve material, and a ring-opening reaction occurs between the ethylene oxide group of the double-bond reagent (i.e., the first functional monomer) and the hydroxyl groups, carboxyl groups as well as the remaining small amount of amino groups after glutaraldehyde crosslinking on the glutaraldehyde crosslinked biological valve material, thereby introducing the first carbon-carbon double bond on the glutaraldehyde crosslinked biological valve material. Further, the second carbon-carbon double bond is introduced through the physical penetration of the second functional monomer. Still further, by initiating the polymerization of these double bonds on the glutaraldehyde crosslinked biological valve material and the functional monomers, the functional monomers are introduced as the crosslinked network to achieve further secondary crosslinking, thereby completing the crosslinking treatment of the biological valve material after double-bond copolymerization.

Since more functional groups (hydroxyl and carboxyl groups other than amino groups) on the biological valve material are used for crosslinking, and the polymers of functional monomers are further introduced as the crosslinked network through copolymerization with functional monomers, the biological valve material has a larger crosslinked network. The crosslinking degree of the biological valve material after crosslinking and double-bond copolymerization will be significantly improved, and its structural stability and anti-calcification performance will also be significantly improved with the introduction of the functional monomer polymer network.

The carbon-carbon double bonds are introduced after crosslinking with glutaraldehyde, they are mainly grafted on the surface of the biological valve material. Since there are no other substances that can participate in the crosslinking reaction added in the process of glutaraldehyde crosslinking modification of the biological valve material, the original fiber structure of the biomaterial can be better protected. This can maintain the original orientation direction of the fibers of the biomaterial while effectively ensuring the mechanical properties of the valve, avoiding the problem of directly adding double-bond functional monomers during crosslinking in previous research, which may damage the original fiber orientation of the biological material and increase fiber disorder.

On the basis of chemically grafting the first carbon-carbon double bond, the second carbon-carbon double bond is further introduced through physical penetration, providing more double bonds as a crosslinking basis for secondary crosslinking, which can further increase the crosslinking degree of the biological valve material and improve the mechanical properties of the biological valve material.

Optionally, in step S120 of the present disclosure, the first carbon-carbon double bond is introduced through a non-condensation chemical bonding.

Optionally, in step S110, the biomaterial does not undergo any chemical reactions involving other reagents before being treated with the aldehyde group crosslinking agent.

Further optionally, in the reaction system of step S120, the first carbon-carbon double bonds are provided by the first functional monomer having the active group, and the reaction raw materials in step S100 only include the biomaterial, the first functional monomer and the aldehyde group crosslinking agent.

In step S110:

The crosslinking agent in the present disclosure is the aldehyde group crosslinking agent used in current mainstream crosslinking methods. Optionally, the aldehyde group crosslinking agent is selected from glutaraldehyde and formaldehyde.

Optionally, the concentration of the glutaraldehyde solution is in the range of 0.1% to 5% (w/w), and the crosslinking time may be any time in the range of 0.5 h to 120 h.

The biomaterials used here are conventional biomaterials used in the existing glutaraldehyde crosslinking processes with collagen content ranging from 60% to 90%. Further, the biomaterials are animal tissues originating from the pig, cattle, horse or sheep, including one or more of the following: pericardium, valve, intestinal valve, meninges, lung valve, blood vessel, skin or ligament.

Optionally, the animal tissue is fresh animal tissue or decellularized biological tissue.

Optionally, in the decellularization, the biological tissue is treated with a surfactant as follows:

the biological tissue is decellularized with an ionic surfactant; or

the biological tissue is decellularized with a non-ionic surfactant.

The ionic surfactant is mainly used to lyse cells, and the non-ionic surfactant is mainly used to remove lipid substances (such as phospholipids).

Optionally, the ionic surfactant is at least one of the following: sodium deoxycholate, fatty acid potassium soap, sodium dodecyl sulfate, sodium cholate, hexadecyltrimethylammonium bromide, fatty acid potassium salt, and alkyldimethylsulfonpropyl betaine.

Optionally, the nonionic surfactant is at least one of Triton and Tween.

In step S120,

optionally, the double-bond reagent, i.e., the first functional monomer, is selected from at least one of allyl glycidyl ether, glycidyl methacrylate and glycidyl acrylate.

Optionally, the concentration of the double-bond reagent in the solution containing the first functional monomer (i.e., the double-bond reagent) is in the range of 1% to 10% (w/w), and the reaction time for the double-bond modification is in the range of 2 h to 120 h.

Optionally, the solvent in the solution containing the first functional monomer, i.e., the double-bond reagent, is one or more of the following: water, physiological saline, neutral pH buffer, or an aqueous solution of methanol, ethanol, ethylene glycol, propanol, 1,2-propylene glycol, 1,3-propylene glycol, isopropanol, butanol, isobutanol, 1,2-butylene glycol, 1,3-butylene glycol, 1,4-butylene glycol, and glycerol.

Optionally, the biofilm material treated in S110 is taken out and washed or directly placed in the solution containing a double-bond reagent (first functional monomer).

In step S130,

the biological valve material treated in step S120 is soaked in the second functional monomer solution directly or after washing.

Optionally, the second functional monomer has at least a second carbon-carbon double bond.

Further optionally, the second functional monomer is one or more of the following: polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-vinylbisacrylamide, (ethane-1,2-diylbis (oxy)) bis (ethane-2,1-diyl) diacrylate, N,N′-dimethylacrylamide, N,N-dimethylmethacrylamide, and double-bonded polylysine.

Optionally, the concentration of the second functional monomer solution is in the range of 0.1% to 20% (v/v); further, the concentration of the second functional monomer solution is in the range of 0.1% to 6% (v/v).

Optionally, the solvent of the second functional monomer solution is one or a mixture of the following: water, physiological saline, ethanol, isopropanol or a neutral pH buffer solution.

Optionally, the immersion time in the second functional monomer solution is in the range of 0.5 h to 120 h.

In step S200,

the biological valve material treated in step S120 is cleaned with deionized water and then soaked in the initiator solution, or the initiator is directly added to the reaction system in step S120 to initiate the polymerization reaction; wherein the latter is commonly known as the one-pot method;

Optionally, the solvent in the solution containing the initiator is water, physiological saline or neutral pH buffer.

Regarding the concentration of the initiator as mentioned above, in the one-pot method, it may be understood as the concentration of the solution of the reaction system in step S120 in the one pot method, while in the step-by-step method, it may be understood as the concentration of the solution containing the initiator.

Optionally, the initiator is a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine; and the concentration of each component in the mixture is in the range of 1 to 100 mM.

The reaction time of step S200 is in the range of 3 to 24 hours.

In the present disclosure, all reactions in S110, S120, S130 and S200 can be carried out at 0 to 50° C. unless otherwise specified. Preferably, the temperature does not need to be specially controlled and can be at room temperature. It is suitable not to exceed the human body's adaptive temperature, preferably in the range of 36 to 37° C.

In the present disclosure, all reactions in S110, S120, S130 and S200 may be either static reactions or dynamic reactions unless otherwise specified. Dynamic reactions may be conducted using equipment capable of circulating the solution, such as a peristaltic pump, or through shaking at a rotation speed ranging from 10 rpm to 150 rpm. The peristaltic cycle or shaking may be carried out continuously or intermittently.

In the present disclosure, optionally, dehydration and drying treatment after the double-bond polymerization is completed are carried out to prepare a dry valve. Further, after the double-bond polymerization is completed, the biological valve material is conventionally cleaned, softened, and then dehydrated and dried.

The cleaning solution may be one or more of water, physiological saline, ethanol, isopropanol or neutral pH buffer solution. The pH may be adjusted to be in the range of 5.0 to 9.5 before and during use, or there is no need to adjust the pH.

Optionally, the dehydration treatment involves exposing the valve piece subjected to double-bond polymerization or the valve formed by sewing valve pieces to a dehydration solution.

Optionally, the dehydration solution is a mixed solution of alcohol solution and water with the alcohol solution accounting for 20% to 90% (v/v). The alcohol reagent may be one or both of ethanol and isopropanol.

Optionally, the drying treatment involves exposing the dehydrated valve piece or valve to a softener solution for 20 minutes to 10 hours.

Optionally, the main component of the softener solution is one or a mixed solution of glycerol and polyethylene glycol, with the glycerol concentration ranging from 10% to 100% (v/v), and the remaining component is one or more of water, ethanol, isopropanol, etc., accounting for 0 to 90% (v/v).

Optionally, the valve sterilization method after drying treatment may be one of ethylene oxide sterilization or electron beam sterilization.

The biological valve material prepared by the above methods may be used for interventional biological valves, for example, through minimally invasive intervention; or the biological valve material prepared by the above methods may be used for surgical biological valves, for example, through surgical implantation.

As shown in FIG. 7, a prosthetic heart valve according to an embodiment includes a stent 1 and leaflets 2 connected in the stent 1. The stent is generally cylindrical, and the side wall thereof has a hollow mesh structure. There is a blood flow channel defined in the stent, and the leaflets cooperate with each other to control the opening and closing of the blood flow channel in the stent.

Depending on the release modes, the stent is made of corresponding materials during processing, such as nickel-titanium alloy that has shape memory and can self-expand in the human body, or stainless steel that is released by ball expansion, etc. The stent itself may be formed by cutting pipes or braiding wires. The leaflets may be connected to the stent by sewing, bonding or integral molding.

For positioning in the human body, positioning structures that can interact with surrounding native tissues may be provided on the periphery of the stent, such as anchors, arms, etc. In order to prevent peripheral leakage, skirts or anti-peripheral leakage materials or the like may be provided on the inside and/or outside of the stent. The leaflets, skirts or anti-peripheral leakage materials may all be made of the biological valve materials in the above embodiments.

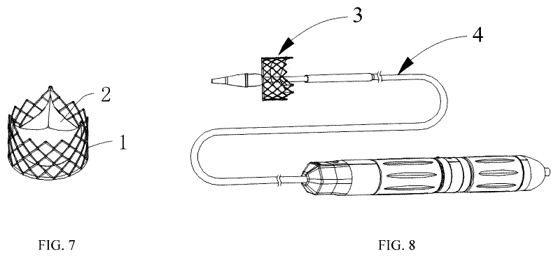

As shown in FIG. 8, in case of intervention through catheter, the prosthetic heart valve 3 and a corresponding delivery system form a valve intervention system. The delivery system includes a catheter assembly 4 and a handle for controlling the catheter assembly. The prosthetic heart valve is in a radially compressed state during delivery in the human body, and is radially expanded in the human body after releasing the restriction of the catheter assembly or being expanded by ball expansion.

Specific examples are illustrated in the following.

### Control Group 1

During the treatment process, a simple glutaraldehyde crosslinking group was set as a control group, and the porcine pericardium was soaked in a 0.625% (w/w) glutaraldehyde solution at room temperature for 72 hours to prepare glutaraldehyde crosslinked porcine pericardium, which was recorded as control group 1.

### Example 1

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water, and then soaked in an isopropanol aqueous solution of 5% (v/v) glycidyl methacrylate at room temperature for double-bond modification of the glutaraldehyde crosslinked porcine pericardium. The reaction time was 72 hours, and the solvent of the double-bond solution was a 20% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 2% (v/v) polyethylene glycol diacrylate solution for 2 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of polyethylene glycol diacrylate. After reacting at 37° C. for 8 hours, a porcine pericardium crosslinked after double-bond copolymerization was obtained, which was recorded as sample 1.

### Example 2

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an aqueous solution of 6% (v/v) propanol glycidyl acrylate at room temperature for double-bond modification of the glutaraldehyde crosslinked porcine pericardium. The reaction time was 72 hours, and the solvent of the double-bond solution used was a 20% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 2.5% (v/v) N-methyl-2-acrylamide solution for 1 hour.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of N-methyl-2-acrylamide. After reacting at 37° C. for 8 hours, a porcine pericardium crosslinked after double-bond copolymerization was obtained, which was recorded as sample 2.

### Example 3

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution with 2% (v/v) glycidyl acrylate and 4% (v/v) allyl glycidyl ether at room temperature for double-bond modification of the glutaraldehyde-crosslinked porcine pericardium. The reaction time was 72 hours, and the solvent of the double-bond solution was a 30% (v/v) aqueous ethanol solution.

After the double-bond modification was completed, the double-bond glutaraldehyde crosslinked porcine pericardium was washed with deionized water; and then the double-bond glutaraldehyde crosslinked porcine pericardium was soaked in a 2.5% (v/v) (ethane-1,2-diylbis(oxy))bis(ethane-2,1-diyl)diacrylate solution for 1 hour.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of (ethane-1,2-diylbis(oxy))bis(ethane-2,1-diyl)diacrylate. After reacting at 37° C. for 8 hours, a porcine pericardium crosslinked after double-bond copolymerization was obtained, which was recorded as sample 3.

### Example 4

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution with 3% (v/v) glycidyl methacrylate and 2% (v/v) glycidyl acrylate at room temperature for double-bond modification of the glutaraldehyde crosslinked porcine pericardium. The reaction time was 48 hours, and the solvent of the double-bond solution was a 25% (v/v) isopropanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.5% (v/v) ethane-1,2-diyl diacrylate solution for 1 hour.

An initiator which included ammonium persulfate at the concentration of 20 mM and sodium bisulfite at the concentration of 10 mM was added to the above solution to further initiate the polymerization between the double bonds on the double-bond glutaraldehyde crosslinked biological valve material and the double bonds of ethane-1,2-diyl diacrylate. After reacting at 37° C. for 7 hours, a porcine pericardium crosslinked after double-bond copolymerization was obtained, which was recorded as sample 4.

### Example 5

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde group crosslinking treatment on the porcine pericardium to prepare glutaraldehyde crosslinked porcine pericardium.

The glutaraldehyde crosslinked porcine pericardium was washed with deionized water and soaked in an ethanol aqueous solution of 4% (v/v) glycidyl methacrylate at room temperature for double-bond modification. The reaction time was 72 hours and the solvent of the double-bond solution was a 20% (v/v) ethanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.4% (v/v) N,N′-dimethylacrylamide solution for 1 hour.

An initiator which included sodium persulfate at the concentration of 20 mM and sodium bisulfite at the concentration of 7 mM was added to the above solution to further initiate polymerization between the double bonds on the double-bond glutaraldehyde crosslinked biological valve material and the double bonds of N,N′-dimethylacrylamide. After reacting at 37° C. for 7 hours, a porcine pericardium crosslinked after double-bond copolymerization was obtained, which was recorded as sample 5.

### Example 6

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isobutanol aqueous solution of 4% (v/v) glycidyl methacrylate at room temperature for double-bond modification of the glutaraldehyde crosslinked porcine pericardium. The reaction time was 72 hours, and the solvent of the double-bond solution was a 15% (v/v) isobutanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.4% (v/v) N,N′-dimethylmethacrylamide solution for 5 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of N,N′-dimethylmethacrylamide. After reacting at 37° C. for 12 hours, a porcine pericardium crosslinked after double-bond copolymerization was obtained, which was recorded as sample 6.

### Example 7

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 4% (v/v) glycidyl acrylate at room temperature for double-bond modification. The reaction time was 48 hours and the solvent of the double-bond solution was 20% (v/v) methanol aqueous solution.

After the double-bond modification was completed, the double-bond glutaraldehyde crosslinked porcine pericardium was washed with deionized water; and then the double-bond glutaraldehyde crosslinked porcine pericardium was soaked in a solution containing 1.0% (v/v) N,N′-dimethylacrylamide and 0.5% (v/v) N,N′-dimethylmethacrylamide for 1 hour.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of N,N′-dimethylacrylamide and N,N′-dimethylmethacrylamide. After reacting at 37° C. for 8 hours, a porcine pericardium crosslinked after double-bond copolymerization was obtained, which was recorded as sample 7.

### Example 8

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an ethylene glycol aqueous solution of 5% (v/v) glycidyl methacrylate at room temperature for double-bond modification of the glutaraldehyde crosslinked porcine pericardium. The reaction time was 72 hours, and the solvent of the double-bond solution was a 25% (v/v) ethylene glycol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.25% (v/v) N,N′-dimethylmethacrylamide solution for 5 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds of N,N′-dimethylmethacrylamide. After reacting at 37° C. for 12 hours, a porcine pericardium crosslinked after double-bond copolymerization was obtained, which was recorded as sample 8.

### Example 9

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in a propanol aqueous solution of 7% (v/v) glycidyl acrylate at room temperature for double-bond modification. The reaction time was 60 hours, and the solvent of the double-bond solution was a 30% (v/v) propanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 1.0% (v/v) N-ethylacrylamide solution for 5 hours.

Initiators which included ammonium persulfate at a concentration of 20 mM and tetramethylethylenediamine at a concentration of 1 mM to the above solution to further initiate polymerization between the double bonds on the double-bond glutaraldehyde crosslinked biological valve material and the double bonds of N,N′-dimethylmethacrylamide. After reacting at 37° C. for 12 hours, a porcine pericardium crosslinked after double-bond copolymerization was obtained, which was recorded as sample 9.

### Example 10

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was washed with deionized water and soaked in an isopropanol aqueous solution containing 4% (v/v) glycidyl methacrylate and 2% (v/v) glycidyl acrylate at room temperature for double-bond modification. The reaction time was 84 hours, and the solvent of the double-bond solution was a 25% (v/v) ethanol aqueous solution.

After the double-bond modification was completed, the double-bond glutaraldehyde crosslinked porcine pericardium was washed with deionized water; then, the double-bond glutaraldehyde crosslinked porcine pericardium was soaked in a 1.50% (v/v) N,N′-dimethylmethacrylamide solution for 3 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds of N,N′-dimethylmethacrylamide. After reacting at 37°° C. for 7 hours, a porcine pericardium crosslinked after double-bond copolymerization was obtained, which was recorded as sample 10.

Performance Characterization of Samples of Examples 1 to 10 and Control Example 1:

In order to characterize the change in the crosslinking degree of glutaraldehyde crosslinked biological valve materials before and after double-bond polymerization and crosslinking treatment, the thermal stability and crosslinking degree of biological valve materials were characterized by measuring the thermal shrinkage temperature of biological valve materials; the stability of biological valve materials was characterized through enzyme degradation experiment; the degree of calcification (anti-calcification performance) of the samples were characterized through rat subcutaneous implantation experiment, and the elastic angle of the biological valve material was tested to characterize its elasticity.

Thermal Shrinkage Temperature Determination:

The biological valve material was cut into a circular sheet with a diameter of 0.6 cm, which was placed in a crucible after drying, and then the thermal shrinkage temperature of the biological valve material was measured on a differential scanning calorimeter in the range of 40° C. to 120° C. at a heating rate of 10° C./min. Thermal stability and crosslinking degree of the biological valve material were characterized by measuring the thermal shrinkage temperature. The higher the thermal shrinkage temperature, the higher the thermal stability and crosslinking degree.

TABLE 1
Thermal shrinkage temperature of biological valve materials

| | Thermal shrinkage |
|---|---|
| Sample | temperature (° C.) |
| Control group 1 (glutaraldehyde | 85.2 |
| crosslinked porcine pericardium) | |
| Sample 1 | 91.3 |
| Sample 2 | 92.8 |
| Sample 3 | 90.7 |
| Sample 5 | 91.4 |
| Sample 10 | 90.1 |

Through the thermal shrinkage temperature measurement of sample 1, sample 2, sample 3, sample 5, sample 10 and the control group 1 (glutaraldehyde crosslinked porcine pericardium), it can be seen that, as shown in Table 1, the thermal shrinkage temperatures of sample 1, sample 2, sample 3, sample 5, sample 10 are all higher than that of control group 1 (glutaraldehyde crosslinked porcine pericardium), that is, the thermal stability and crosslinking degree of sample 1, sample 2, sample 3, sample 5, sample 10 were all higher than those in the control group 1 (glutaraldehyde crosslinked porcine pericardium). The experimental results of the thermal shrinkage temperature measurement show that the method for preparing biological valve materials by crosslinking after double-bond copolymerization of the present disclosure can improve the thermal stability and crosslinking degree of biological valves.

### Elasticity Test Experiment

The biological valve material with uniform thickness was cut into a rectangular sample of 1*4.6 cm², which was clamped horizontally along the midline of the long side thereof. The angle at which the sample sag relative to the horizontal plane of the midline was tested to characterize the elasticity of the sample. The smaller the angle, the higher the elasticity.

TABLE 2
Elastic angle of biological valve materials

| Sample | Elastic angle (°) |
|---|---|
| Control group 1 (glutaraldehyde | 65 |
| crosslinked porcine pericardium) | |
| Sample 1 | 35 |
| Sample 2 | 45 |
| Sample 3 | 56 |
| Sample 5 | 47 |
| Sample 10 | 50 |

The elasticity test experiments were conducted on sample 1, sample 2, sample 3, sample 5, sample 10 and control group 1 (glutaraldehyde crosslinked porcine pericardium) to characterize their elasticity. The results of the elasticity test are shown in Table 2. Compared with control group 1 (glutaraldehyde crosslinked porcine pericardium), the elastic angles of sample 1, sample 2, sample 3, sample 5, sample 10were lower, indicating that their elasticity was significantly improved than the control group (glutaraldehyde crosslinked porcine pericardium). The method of preparing biological valve materials by crosslinking after double-bond copolymerization can improve the elasticity of biological valve materials, which is conducive to rapid recovery of shape after transcatheter implantation.

### Enzyme Degradation Experiment

The obtained biological valve material was cut into circular sheets with a diameter of 1 cm, with each group consisting of 6-8 parallel test samples. All circular sheet samples were placed on a 48-well plate, frozen overnight at −80° C., and then transferred to a vacuum freeze-dryer to freeze-dry for 48 h. Each sample was weighed on a microbalance as the initial weight (Wo) and placed back into the 48-well plate. 0.5 mL collagenase I solution in PBS was added with a pipette to the wells of the 48-well plate so that the biological valve sample is completely soaked in the collagenase solution in PBS (100 U/mL). Then the 48-well plate was transferred to a 37° C. constant temperature incubator for incubation for 24 h. After the incubation, the biological valve material sample was taken out. After repeated flushing three times, the sample was frozen overnight at −80° C. and then transferred to a vacuum freeze-dryer to freeze-dry for 48 h. Each sample after degradation by collagenase solution was weighed on a microbalance as the final weight (Wt). The calculation formula for enzymatic degradation weight loss rate is as follows:

$$Enzyme degradation weight loss rate = \frac{{w}_{0} - {w}_{t}}{{w}_{0}} \times 100 \% .$$

The collagenase degradation weight loss rate was measured for sample 1, sample 2, sample 5, sample 10 and control group 1. The results are shown in Table 3.

TABLE 3
Enzyme degradation weight loss rate of biological valve materials

| | Enzyme degradation |
|---|---|
| Sample | weight loss rate (%) |
| Control group 1 (glutaraldehyde | 7.27 ± 1.18 |
| crosslinked porcine pericardium) | |
| Sample 1 | 1.12 ± 0.40 |
| Sample 2 | 3.61 ± 0.22 |
| Sample 5 | 2.90 ± 0.45 |
| Sample 10 | 3.27 ± 0.55 |

The enzyme degradation experiments were conducted on sample 1, sample 2,sample 5, sample 10 and control group 1 (glutaraldehyde crosslinked porcine pericardium) to characterize the crosslinking degree of samples, and the enzymatic degradation weight loss rates of samples after sample 1, sample 2, sample 5, sample 10and control group 1 were treated with collagenase I were then calculated as shown in Table 3. The enzymatic degradation weight loss rates of sample 1, sample 2, sample 5,sample 10 were all lower than that of the control group 1 (glutaraldehyde crosslinked porcine pericardium), which indicates that sample 1, sample 2, sample 5, sample 10 have higher stability than the control group (glutaraldehyde crosslinked porcine pericardium), that is, sample 1, sample 2, sample 5, sample 10 are more stable. The results of the enzyme degradation experiment show that the method for preparing biological valve materials by crosslinking after double-bond copolymerization of the present disclosure can improve the stability of biological valve materials.

### Anti-calcification Test

The biological valve materials were cut into 1*1 cm² sheets, sterilized and implanted subcutaneously in rats for 30 days before being taken out. Each sample was divided into two parts. One part was decapsulated and freeze-dried and weighed, and then digested with 6 M hydrochloric acid to measure the calcium content per gram of sample. The other part of the sample was fixed with paraformaldehyde tissue fixative. After fixation, the sampled were taken out and smoothed with a scalpel, and then transferred to a dehydration box. The material samples were dehydrated using gradient ethanol. After dehydration, the material samples were transferred to an embedding machine and embedded in melted wax, and then transferred to a −20°° C. refrigerator to cool and trimmed. Slices with 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

The biological valve materials of sample 1, sample 2, sample 5 and control group 1 (glutaraldehyde crosslinked porcine pericardium) were cut into 1*1cm² sheets for anti-calcification tests.

TABLE 4
Calcium content of biological valve materials after
subcutaneous implantation in rats for 30 days

| Sample | Calcium content (mg/g) |
|---|---|
| Control group 1 (glutaraldehyde | 67.3 ± 10.5 |
| crosslinked porcine pericardium) | |
| Sample 1 | 5.4 ± 2.7 |
| Sample 2 | 8.1 ± 3.6 |
| Sample 5 | 13.9 ± 4.7 |

The calcium contents of sample 1, sample 2, sample 5 and control group 1 (glutaraldehyde crosslinked porcine pericardium) after being implanted subcutaneously in rats for 30 days was measured to characterize the degree of calcification of samples. As shown in Table 4, the calcium contents of sample 1, sample 2, sample 5 and control group 1 after being implanted subcutaneously in rats for 30 days were all lower than that of the control group (glutaraldehyde crosslinked porcine pericardium). This result indicates that the method for preparing biological valve materials by crosslinking after double-bond copolymerization can improve the anti-calcification performance of biological valves.

Alizarin red Staining Experiment:

Sample 1, sample 2, sample 5 and control group 1 were implanted into the subcutaneous tissue of rats for 30 days and then taken out and fixed with paraformaldehyde tissue fixative. After fixation, the sampled were taken out and smoothed with a scalpel, and then transferred to a dehydration box. The material samples were dehydrated using gradients of 50%, 75%, 85%, 95% (v/v) and absolute ethanol. After dehydration, the material samples were transferred to an embedding machine and embedded in melted wax, and then transferred to a −20° C. refrigerator to cool and trimmed. Slices with 3 to 5 μm thickness were obtained from the trimmed wax blocks with a slicer, and transferred from the sheeter to a glass slide, and dewaxed and rehydrated. The slices were stained with alizarin red staining solution for 3 minutes, washed with water, dried and permeabilized with xylene for 5 minutes. The slices were sealed with neutral gum and images of the staining results were collected through a pathology slide scanner.

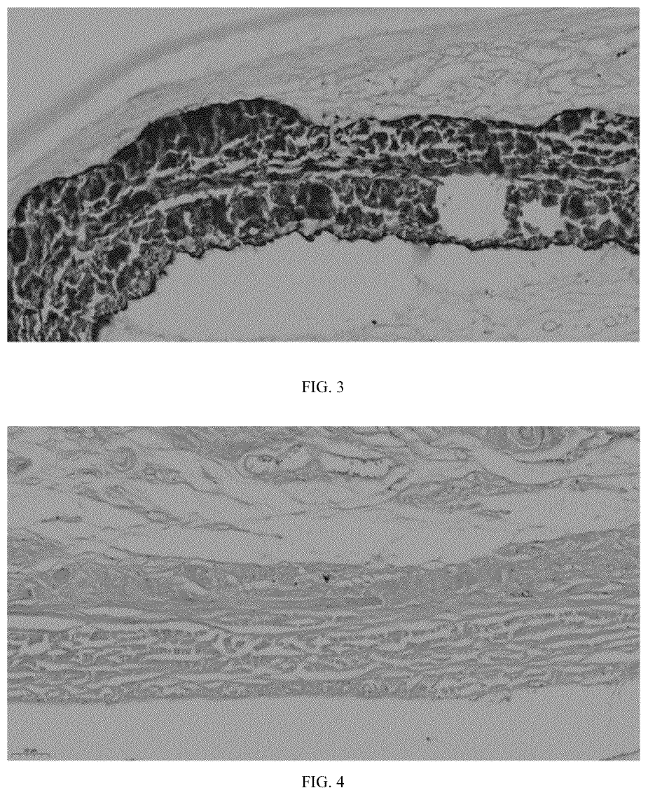

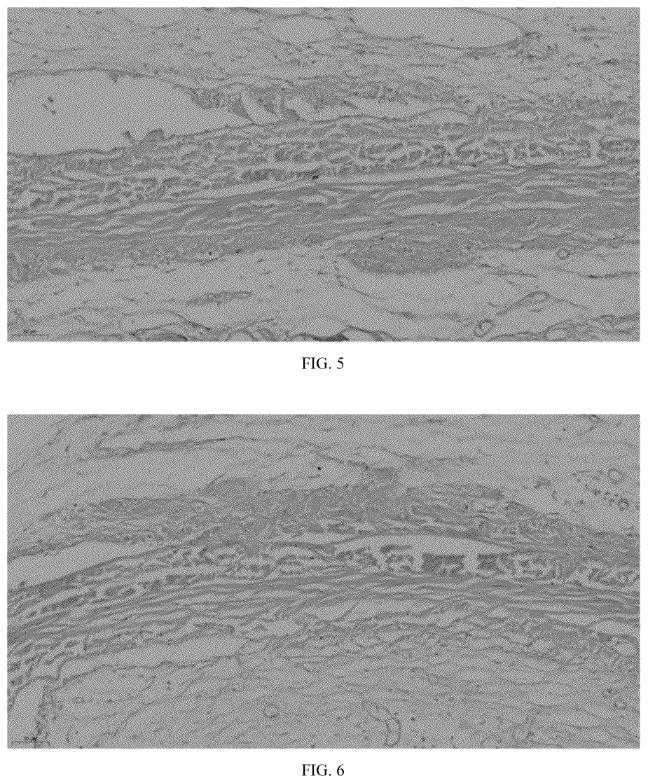

The alizarin red staining experiment was conducted on control group 1(glutaraldehyde crosslinked porcine pericardium), sample 1, sample 2, and sample 5 after being implanted subcutaneously in rats for 30 days to directly observe the degree of calcification of samples. Images of the alizarin red staining results of sample slices after being implanted subcutaneously in rats for 30 days are shown in FIGS. 3 to 12, wherein the darker the color of the sample after alizarin red staining, the higher the degree of calcification. Compared with the alizarin red staining result of slice of control sample 1 (glutaraldehyde crosslinked porcine pericardium) (FIG. 3), the color of the alizarin red staining results of slices of sample 1 (FIG. 4), sample 2 (FIG. 5), and sample 5 (FIG. 6) is obviously lighter, which directly indicates that the degrees of calcification of sample 1, sample 2, and sample 5 are lower than that of the control group 1, that is, compared with control group 1, sample 1, sample 2, and sample 5 have stronger anti-calcification effect. The alizarin red staining results of biological valve materials after being implanted subcutaneously in rats for 30 days show that the method for preparing biological valve materials by crosslinking after double-bond copolymerization of the present disclosure can improve the anti-calcification performance of biological valves.

### Example 11

Freshly collected porcine pericardium was soaked in physiological saline and washed by shaking for 2 hours, and then soaked in a 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 5% (v/v) glycidyl methacrylate at room temperature for double-bond modification of the glutaraldehyde crosslinked porcine pericardium. The reaction time was 72 hours, and the solvent of the double-bond solution was a 18% (v/v) isopropanol aqueous solution.

After the double-bond modification was completed, the double-bond glutaraldehyde crosslinked porcine pericardium was washed with deionized water; and then the double-bond glutaraldehyde crosslinked porcine pericardium was soaked in a 2% (v/v) polyethylene glycol diacrylate solution for 2 hours.

An initiator which included ammonium persulfate at a concentration of 20 mM and sodium bisulfite at a concentration of 10 mM was added to the above solution to further initiate polymerization between the double bonds on the double-bond glutaraldehyde crosslinked biological valve material and the double bonds on polyethylene glycol diacrylate, and after reacting at 37° C. for 8 hours, a porcine pericardium crosslinked after double-bond copolymerization was obtained.

The porcine pericardium material crosslinked after double-bond copolymerization was placed in a 60% isopropanol aqueous solution for 45 minutes and then placed in a solution containing 10% glycerol, 3% polyethylene glycol (Mn=200) and 87% ethanol for 3 hours at room temperature. The excess glycerol was removed from the surface of the porcine pericardium material, which was sterilized with ethylene oxide, and recorded as sample 11.

### Example 12

Fresh porcine pericardium was placed in PS solution containing 0.5% sodium deoxycholate (surfactant) by mass, shaken for 4 hours at room temperature, and then washed with a sodium chloride aqueous solution (i.e. physiological saline) with a mass fraction of 0.9% three times.

The porcine pericardium was immersed in 0.625% (w/w) glutaraldehyde solution at room temperature and shaken for 72 hours to perform glutaraldehyde crosslinking treatment on the porcine pericardium to prepare glutaraldehyde crosslinked porcine pericardium.

The glutaraldehyde crosslinked porcine pericardium was further washed with deionized water and soaked in an isopropanol aqueous solution of 5% (v/v) glycidyl methacrylate at room temperature for double-bond modification of the glutaraldehyde crosslinked porcine pericardium. The reaction time was 72 hours, and the solvent of the double-bond solution was a 18% (v/v) isopropanol aqueous solution.

After the double-bond modification was completed, the double-bond glutaraldehyde crosslinked porcine pericardium was washed with deionized water; then, the double-bond glutaraldehyde crosslinked porcine pericardium was soaked in a 2% (v/v) polyethylene glycol diacrylate solution for 2 hours.

An initiator which included ammonium persulfate at a concentration of 20 mM and sodium bisulfite at a concentration of 10 mM was added to the above solution to further initiate the polymerization between the double bonds on the double-bond glutaraldehyde crosslinked biological valve material and the double bonds on polyethylene glycol diacrylate. After reacting at 37° C. for 8 hours, a porcine pericardium crosslinked after double-bond copolymerization was obtained, which was recorded as sample 12.

The above-described embodiments only illustrate several embodiments of the present disclosure, and the description thereof is specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure. Therefore, the protection scope of the present disclosure should be determined by the appended claims.

What is claimed is:

1. A method for preparing a biological valve material based on aldehyde group crosslinking, comprising:
step S110: contacting biomaterial with an aldehyde group crosslinking agent solution for crosslinking;
step S120: soaking the biomaterial treated in step S110 in a solution containing a first functional monomer to chemically introduce a first carbon-carbon double bond, wherein the first functional monomer has the first carbon-carbon double bond and an ethylene oxide group;
step S130: soaking the biomaterial treated in step S120 in a solution containing a second functional monomer, wherein the second functional monomer has a second carbon-carbon double bond; and
step S200, performing a polymerization reaction of carbon-carbon double bonds to obtain the biological valve material under an action of an initiator.

2. The preparation method according to claim 1, wherein the aldehyde group crosslinking agent is glutaraldehyde or formaldehyde.

3. The preparation method according to claim 1, wherein the biological material is an animal tissue originating from one or more of the following: pericardium, valve, intestinal valve, meninges, lung valve, blood vessel, skin or ligament.

4. The preparation method according to claim 3, wherein the animal tissue is a fresh animal tissue or a decellularized biological tissue.

5. The preparation method according to claim 1, wherein step S200 comprising:
adding the initiator to a system treated in a previous step; or taking the biomaterial treated in the previous step out and directly or after washing soaking the biomaterial treated in the previous step in a solution containing the initiator.

6. The preparation method according to claim 1, wherein in step S200, the initiator is a single initiator or a mixed initiator, and a polymerization time is in a range of 3 to 24 hours.

7. The preparation method according to claim 6, wherein the mixed initiator is:
a mixture of ammonium persulfate and sodium bisulfite, or a mixture of ammonium persulfate and sodium sulfite, or a mixture of sodium persulfate and sodium sulfite, or a mixture of potassium persulfate and sodium sulfite, or a mixture of sodium persulfate and sodium bisulfite, or a mixture of potassium persulfate and sodium bisulfite, or a mixture of potassium persulfate and tetramethylethylenediamine, or a mixture of ammonium persulfate and tetramethylethylenediamine, or a mixture of sodium persulfate and tetramethylethylenediamine;
and a concentration of each component in the mixture is in a range of 1 to 100 mM.

8. The preparation method according to claim 7, wherein the single initiator is any component of the mixed initiator.

9. The preparation method according to claim 1, wherein the first functional monomer is at least one selected from a group consisting of allyl glycidyl ether, glycidyl methacrylate and glycidyl acrylate.

10. The preparation method according to claim 1, wherein in step S110, a w/w concentration of the aldehyde group crosslinking agent solution is in a range of 0.1% to 5%; and a crosslinking time is in a range of 0.5 h to 120 h.

11. The preparation method according to claim 1, wherein in step S120, a w/w concentration of the first functional monomer in the solution containing the first functional monomer is in a range of 1% to 10%; a reaction time is in a range of 2 to 120 hours, and the solution containing the first functional monomer only contains the first functional monomer and a solvent that does not participate in the chemical reaction.

12. The preparation method according to claim 1, wherein a solvent in the solution containing the first functional monomer is one or more of the following: water, physiological saline, a neutral pH buffer, and an aqueous solution of any one of methanol, ethanol, ethylene glycol, propanol, 1,2-propanediol, 1,3-propanediol, isopropanol, butanol, isobutanol, 1,2-butanediol, 1,3-butanediol, 1,4-butanediol, and glycerol.

13. The preparation method according to claim 1, wherein the second functional monomer is selected from one or more of the following: polyethylene glycol diacrylate, 1,4-butanediol diacrylate, ethane-1,2-diyl diacrylate, ethyl acrylate, N-methyl-2-acrylamide, N-2,2-propenyl-2-acrylamide, N-ethylacrylamide, N,N′-vinylbisacrylamide, (ethane-1,2-diylbis(oxy))bis(ethane-2,1-diyl)diacrylate, N,N′-dimethylacrylamide, N,N-dimethylmethacrylamide, and double-bond polylysine.

14. The preparation method according to claim 1, wherein in step S130, a v/v concentration of the second functional monomer in the solution containing the second functional monomer is in a range of 0.1% to 20%; and an immersion time is in a range of 0.5 h to 120 h.

15. The preparation method according to claim 1, wherein the second functional monomer permeates the biomaterial by physical penetration, a v/v concentration of the second functional monomer in the solution containing the second functional monomer is in a range of 0.1% to 6%, and the solution containing the second functional monomer only contains the second functional monomer and a solvent that does not participate in the reaction.

16. The preparation method according to claim 1, wherein the solvent in the solution containing the second functional monomer is one or a mixture of the following: water, physiological saline, ethanol, isopropanol or a neutral pH buffer solution.

17. A biological valve material prepared by the preparation method according to claim 1.

18. A biological valve, comprising a stent and leaflets, wherein the leaflets are made of the biological valve material according to claim 17.

19. The biological valve according to claim 18, wherein the biological valve is a prosthetic heart valve.

20. An interventional system, comprising a prosthetic heart valve and a catheter assembly, wherein the prosthetic heart valve is delivered by the catheter assembly after being folded, and wherein the prosthetic heart valve comprises a stent and leaflets, and the leaflets are made of the biological valve material according to claim 17.
